(19) United States
(12) Patent Application Publication
(10) Pub. No.: US 2004/0146474 A1
(43) Pub. Date: Jul. 29, 2004

(54) Method for softening lines and relaxing the skin with adenosine and adenosine analogues

(75) Inventor: Jean-Baptiste Galey, Aulnay-Sous-Bois (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/701,495

(22) Filed: Nov. 6, 2003

Related U.S. Application Data
(60) Provisional application No. 60/432,634, filed on Dec. 12, 2002.

(30) Foreign Application Priority Data
Nov. 26, 2002 (FR) 0214828

(51) Int. Cl. A61K 7/06; A61K 7/11; A61K 31/7076

(52) U.S. Cl. 424/70.13; 514/47

(57) ABSTRACT

The present invention concerns a method for softening lines and/or relaxing the skin with adenosine and/or an analogue of adenosine.

### REFERENCE TO PRIOR APPLICATIONS

This application claims priority to U.S. provisional application 60/432,634 filed Dec. 12, 2002, and to French patent application 0214828 filed Nov. 26, 2002, both incorporated herein by reference.

### FIELD OF THE INVENTION

The present invention relates to a method for softening lines and/or relaxing the skin, and/or relaxing facial features, comprising topical application to the skin of a composition comprising at least one compound selected from the group consisting of adenosine and analogues of adenosine, in a physiologically acceptable medium. Particular uses of the invention composition include the decreasing of wrinkles, the reduction in laugh lines, the reduction in frown lines, etc.

It also relates to the use of at least one compound as defined above, in a composition suitable for topical application to the skin, as an agent intended to soften lines and/or relax the skin and/or relax facial features.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women and, increasingly, men have a tendency to want to appear young for as long as possible, and so they seek to tone down signs of ageing in the skin, primarily wrinkles and fine lines. Thus, advertisements and the fashion industry promote products intended to keep the skin radiant and wrinkle-free, the trade marks of a young skin, for as long as possible. Furthermore, physical appearance has an effect on psyche and/or morale.

Until now, wrinkles and fine lines have been treated using cosmetic products containing active ingredients that have an effect on the skin, for example by moisturizing it or improving cell renewal, or by encouraging the synthesis of collagen from which cutaneous tissue is formed, or by preventing its degradation.

Although such treatments can have an effect on wrinkles and fines lines due to chronological or intrinsic ageing, and on those cells due to photo-ageing, they do not have any effect on expression lines.

Expression lines are produced by mechanisms that differ from those generating lines due to ageing.

More precisely, they are produced by the stress exerted on the skin by the facial muscles which produce facial expressions. Depending on the shape of the face, the frequency of expressions and the existence of any tics, they can appear in childhood. Age and some environmental factors such as exposure to the sun do not have any effect on their genesis but can make them deeper and render them permanent.

Expression lines are characterized by the presence of furrows at the periphery of the orifices, namely the nose (nasogenic furrows), the mouth (parabuccal lines and bitterness lines) and the eyes (crows feet) around which the facial muscles are located, and also between the eyebrows (glabellar lines or frown lines) and on the forehead.

Until now, the only routine means for dealing with expression lines are botulinum toxin, which is injected into the glabellar lines (see J. D. Carruthers et al, J. Dermatol. Surg. Oncol., 1992, 18, pp 17-21) and degradable collagen-based, hyalruonic acid-based or polylactic acid-based implants.

Further, as an alternative to those medical techniques requiring the services of a skilled practician, the Applicant has proposed a number of compounds that can provide a myorelaxing effect when topically applied to the skin and which allow expression lines to be dealt with in a different manner. Examples of such compounds that can be cited are antagonists for receptors associated with calcium channels (French application FR-A-2 793 681), and in particular manganese and its salts (FR-A-2 809 005) and alverine (FR-A-798 590); and agonists for receptors associated with the chlorine channel, including glycine (EP-A-0 704 210) and certain extracts of Iris pallida (FR-A-2 746 641).

However, there is still a need for effective compounds for relaxing the skin with a view to smoothing or toning down expression lines.

### BRIEF DESCRIPTION OF THE FIGURE

FIG. 1 illustrates the contraction over time of an equivalent dermis treated with adenosine.

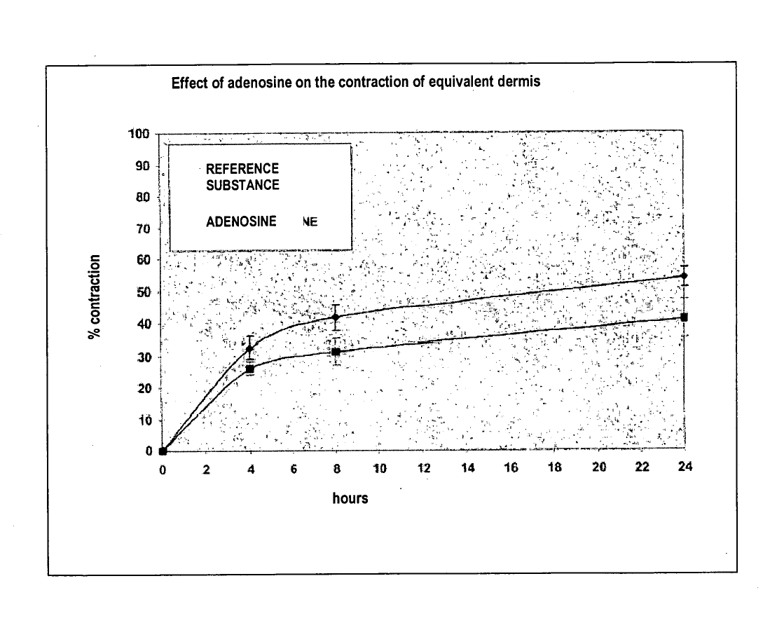

### DETAILED DESCRIPTION OF THE INVENTION

As noted above, the present invention relates to a method for softening lines and/or relaxing the skin, and/or relaxing facial features, comprising topical application to the skin of a composition comprising at least one compound selected from the group consisting of adenosine and analogues of adenosine, in a physiologically acceptable medium. Particular uses of the invention composition include the decreasing of wrinkles, the reduction in laugh lines, the reduction in frown lines, etc.

The inventor has surprisingly discovered that adenosine and its analogues can satisfy the above need for effective compounds for relaxing the skin with a view to smoothing or toning down expression lines, relaxing the skin, relaxing facial features, decreasing wrinkles, reducing laugh lines, reducing frown lines, etc. More precisely, the inventor has demonstrated that adenosine and its analogues can relax the dermal contractile cells which are believed to be involved in the genesis of expression lines, etc. It is believed that the phenotype of certain fibroblasts located along the tension lines created under the effect of contraction of facial muscles when making a facial expression is progressively modified under the effect of said contractions, endowing said fibroblasts with particular contractile properties. Relaxing those cells would thus combat expression lines. Of course, the inventor is not bound by any theory of operation.

In the pharmaceutical field, adenosine is administered orally or intravenously as a vasodilator and an anti-arrythmic.

In the cosmetics field, it has been suggested, in U.S. Pat. No. 6,423,327 and U.S.-2003/044439, that adenosine or an analogue of adenosine be used in a composition that is topically applied to the skin to improve skin condition and in particular to combat lines, skin laxity, skin dryness and pigmentary blemishes. It was indicated that adenosine increases the size of fibroblasts and increases the synthesis of proteins by fibroblasts.

In the same field, documents WO-A-01/43704, U.S. Pat. No. 3,978,213, U.S. Pat. No. 5,371,089, German patents DE-195 45 107 and DE-200 22 691 disclose compositions with an anti-ageing effect comprising adenosine or an adenosine analogue.

None of those documents suggests that adenosine could have a relaxing effect on contractile fibroblasts.

Thus, the present invention provides a method for softening lines and/or relaxing the skin, comprising topical application to the skin of a composition comprising at least one compound selected from adenosine and an analogue of adenosine, in a physiologically acceptable medium.

It also concerns the use of at least one compound as defined above in a composition adapted for topical application to the skin as an agent for softening lines and/or relaxing the skin.

The present invention further provides a method for softening lines and/or relaxing the skin, comprising topical application to the skin of an amount of a composition comprising at least one compound selected from the group consisting of adenosine and analogues of adenosine, in a physiologically acceptable medium, effective to provide a relaxing effect on contractile fibroblasts.

Adenosine analogues that can be used in accordance with the invention and can be cited as particularly useful herein include agonists of adenosine receptors and compounds increasing intra- or extra-cellular adenosine levels.

Examples of adenosine analogues include: 2′-deoxyadenosine; 2′,3′-isopropoylidene adenosine; toyocamycin; 1-methyladenosine, N-6-methyladenosine; adenosine N-oxide; 6-methylmercaptopurine riboside; 6-chloropurine riboside; 5′-adenosine monophosphate; 5′-adenosine diphosphate and 5′-adenosine triphosphate.

Other adenosine analogues include agonists of adenosine receptors, including phenylisopropyl adenosine (PIA), 1-methylisoguanosine, N⁶-cyclohexyl adenosine (CHA), N⁶-cyclopentyl adenosine (CPA), 2-chloro-N-6-cyclopentyladenosine, 2-chloroadenosine, N⁶-phenyladenosine, 2-phenylaminoadenosine, MECA, N⁶-phenethyladenosine, 2-p-(2-carboxyethyl)-phenethyl-amino-5′-N-ethylcarboxamido-adenosine (CGS-21680), N-ethylcarboxamido-adenosine (NECA), 5′-(N-cyclopropyl)-carboxamidoadenosine, DPMA (PD 129,944) and metrifudil.

Other adenosine analogues include compounds which increase the intracellular concentration of adenosine such as erythro-9-(2-hydroxy-3-nonyl) adenine (EHNA) and iodotubercidin.

Other adenosine analogues include salts and esters of adenosin.

Adenosine is preferred for use in the present invention. It is commercially available in the form of a powder from PHARMA WALDHOF.

The composition in accordance with the invention is preferably intended to be applied to zones of the face or forehead marked with expression lines and/or to persons having expression lines.

The lines concerned are preferably selected from: crow's feet, nasogenic furrows, inter-eyebrow lines and forehead lines.

The quantity of adenosine and/or adenosine analogue for use in accordance with the invention is a function of the desired effect and can thus vary widely. To provide an order of magnitude, the composition of the invention can comprise 0.001% to 10% by weight, preferably 0.01% to 1% by weight of adenosine and/or adenosine analogue with respect to the total composition weight.

The composition of the invention is suitable for topical application to the skin and thus it contains a physiologically acceptable medium, i.e. a medium that is compatible with the skin. Such media can comprise water, C1-C8, preferably C1-C4, alcohols, etc.

This composition can be fluid to a greater or lesser extent and can have the appearance of a white or coloured cream, a pommade, milk, serum, paste or foam. It can also be in the form of a solid, in particular in the form of a stick. It can be used as a skin care product and/or as a skin makeup product.

The composition of the invention can be in any form, including any of the galenical forms that are normally used in the cosmetics field; in particular, it can be in the form of an aqueous, possibly gelled solution, a lotion type dispersion which may be a two-phased dispersion, an emulsion obtained by dispersing an oily phase in an aqueous phase (O/W) or vice versa (W/O), a triple emulsion (W/O/W or O/W/O) or an ionic and/or nonionic vesicular type dispersion. Said compositions are prepared using the usual methods. Preferably, a composition in the form of an oil-in-water emulsion is used in the present invention.

When the composition used in the invention is an emulsion, the proportion of oily phase can be from 5% to 80% by weight, preferably 5% to 50% by weight with respect to the total composition weight. Oils, emulsifying agents and co-emulsifying agents used in the composition in the emulsion form are selected from those conventionally used in the field under consideration. The emulsifying agent and co-emulsifying agent are present in the composition in a proportion of 0.3% to 30% by weight, preferably 0.5% to 20% by weight with respect to the total composition weight.

Oils that can be used in the invention that can be cited are hydrocarbons of mineral or synthetic origin (Vaseline oil, isohexadecane), oils of plant origin (apricot kernel oil, the liquid fraction of karite butter oil, avocado, soya oil), oils of animal origin (lanolin), synthesized oils (perhydrosqualene, pentaerythrityl tetraoctanoate), silicone oils (cyclopentasiloxane and cyclohexasiloxane) and fluorinated oils (perfluoropolyethers). It is also possible to use fatty alcohols (cetyl alcohol or stearyl alcohol), fatty acids (stearic acid) or waxes (carnauba wax, ozokerite, beeswax) as the oily materials.

Examples of emulsifying and co-emulsifying agents that can be used in the invention that can be cited are esters of fatty acids and polyethylene glycol such as PEG-100 stearate and PEG-20 stearate and esters of fatty acids and glycerin such as glyceryl stearate.

The composition of the invention can also contain adjuvants, including those that are normal in the cosmetics field, such as hydrophilic or lipophilic gelling agents, hydrophilic or lipophilic active ingredients, preservatives, antioxidants, solvents, perfumes, fillers, filters, pigments, odour absorbers and colorants. The quantities of these different adjuvants are those that are conventionally used in the field under consideration, for example 0.01% to 20% of the total composition weight. Depending on their nature, such adjuvants can be introduced into the oily phase, into the aqueous phase or into the lipid vesicles. In all cases, said adjuvants and the proportions thereof should be selected so that they do not deleteriously affect the desired properties of the adenosine/analogue.

Particular examples of hydrophilic gelling agents that can be cited are carboxyvinyl polymers (carbomers), acrylic copolymers such as acrylate/alkylacrylate copolymers, polyacrylamides, polysaccharides, natural gums and clays, and examples of lipophilic gelling agents that can be cited are modified clays such as bentonites, metal salts of fatty acids, hydrophobic silicon and polyethylenes.

Examples of preservatives that can be cited are esters of para-hydroxybenzoic acid, octane-1,2-diol, 3-iodo-2-propynyl-butylcarbamate, phenoxyethanol and chlorhexidine gluconate.

Examples of fillers that can be cited are polyamide (Nylon) particles; polymethyl methacrylate microspheres; ethylene-acrylate copolymer powders; expanded powders such as hollow microspheres and in particular, microspheres formed from a terpolymer of vinylidene chloride, acrylonitrile and methacrylate and sold by Kemanord Plast under the trade name EXPANCEL; powders of natural organic materials such as starch powders, in particular corn starch, wheat starch or rice starch, which may or may not be cross-linked, such as starch powder cross-linked with octenyl succinate anhydride; silicone resin microbeads such as those sold by Toshiba Silicone under the trade name TOSPEARL; silica; metal oxides such as titanium dioxide or zinc oxide; mica; and mixtures thereof.

As indicated above, the composition of the invention can also include UVA and/or UVB filters in the form of organic or inorganic compounds, the latter optionally being coated to render them hydrophobic.

More particularly preferred organic filters are selected from the following compounds (cited using the CTFA nomenclature): Ethylhexyl Salicylate, Ethylhexyl Methoxycinnamate, Octocrylene, Phenylbenzimidazole Sulfonic Acid, Benzophenone-3, Benzophenone-4, Benzophenone-5,4-Methylbenzylidene camphor, Terephthalylidene Dicamphor Sulfonic Acid, Disodium Phenyl Dibenzimidazole Tetra-sulfonate, 2,4,6-tris-(diisobutyl-4′-aminobenzalmalonate)-s-triazine, Anisotriazine, Ethylhexyl triazone, Diethylhexyl Butamido Triazone, Methylene bis-Benzotriazolyl Tetramethylbutylphenol, Drometrizole Trisiloxane, 1,1-dicarboxy-(2,2′-dimethylpropyl)-4,4-diphenylbutadiene and mixtures thereof.

The inorganic filters are preferably constituted by an oxide of zinc, iron, zirconium, cerium and/or titanium (amorphous or crystalline in the form of rutile and/or anatase), preferably of nanometric dimensions (mean primary particle size: generally in the range 5 nm to 100 nm, preferably in the range 10 nm to 50 nm), optionally coated with alumina and/or stearic acid.

The invention will now be illustrated by the following non-limiting examples. In said examples, reference will be made to the accompanying FIGURE which illustrates the contraction over time of an equivalent dermis treated with adenosine.

### EXAMPLES

### Example 1

### Determination of Dermo-Relaxant Effect of Adenosine

a) Principle of the Test

The principle of this test is to study the relaxing effect of adenosine on an equivalent dermis model constituted by a matrix of collagen seeded with normal human fibroblasts.

These conditions were intended to imitate in vitro the dermal contractile phenomena which occur during facial expressions. Under these conditions, cells spontaneously express tensile forces which induce retraction of the collagen gel. This results in a reduction in the total surface area of the equivalent dermis over time. Measuring that surface area allows the relaxation effects of substances that have been brought into contact with the equivalent dermis to be determined.

b) Protocol

Two series of 3 attached equivalent dermises containing normal human fibroblasts were prepared: a control series with no treatment, and a series treated with adenosine (0.01%). The experiment was carried out three times.

The skin equivalents were prepared as described by Asselineau et al, Exp. Cell. Res., 1985, 159, 536-539; Models in Dermatology, 1987, vol 3, pp 1-7, in the following proportions:

| | |
|---|---|
| MEM medium (1.76X) with or | 45% |
| without adenosine: | |
| Foetal calf serum: | 9% |
| NaOH (0.1 N): | 5% |
| Acetic acid (1/1000): | 4% |
| Collagen: | 26% |
| Fibroblasts: | 11% |

The treated equivalent dermis differed from the control equivalent dermis in that 0.01% of adenosine had been added.

The collagen used was type I collagen (commercial solution), but it was also possible to use type III or IV collagen. It was extracted from rat tails or calf skin by acid hydrolysis and stored in an acidic medium at +4° C.; it polymerizes naturally by heating to 37° C. and by reducing the acidity. The collagen had been dialyzed against successive baths of water+acetic acid.

The following protocol was employed: the following were introduced into a sterile tube: 1.76×MEM medium in the presence of additives (glutamine 1%, non essential amino acids 1%, sodium pyruvate 1%, fungizone 1% and penicillin/streptomycin 1%), foetal calf serum, 0.1 N sodium hydroxide NaOH. Fibroblasts isolated from human skin explants were then added in a concentration of 1.4×10⁵ cells per ml of culture medium.

A 1/1000 vol/vol mixture of collagen in acetic acid was then slowly added by pouring it down the tube wall so that the appearance of a whitish cloud was observed.

The ensemble was then carefully mixed and distributed into the wells of a 12-well culture plate (Costar, reference 3512) in an amount of 0.5 ml of mixture per cm². The culture plate was placed in an incubator at 37° C. with 5% CO₂.

Once formed after polymerizing the collagen, the equivalent dermises were left adhering to the culture support for 3 days then detached from the support so that contraction could commence. Said attached equivalent dermises were removed from the incubator to record images with a view to measuring their surface area at each point of the contraction kinetics (0, 4, 8 and 24 hours). They were immediately replaced in the incubator between each measuring point.

The spontaneous contraction of the treated (with adenosine) equivalent dermises and control (no adenosine) equivalent dermises was carried out by measuring their surface area at different times after the onset of spontaneous contraction.

To this end, a digital image was acquired for each treated or untreated equivalent dermis using a camera (CCD Camera—Iris Sony DXC—107P) and the surface area was then calculated for each image using an image analysis system (Zeiss Axiovision 3.0). This surface area measurement corresponded to a percentage contraction which equals the ratio of the surface areas in accordance with the formula:

% contraction=(Sp−Si)/Sp×100

in which:

“Sp” represents the surface area of one well in the culture plate; it corresponds to the total surface area of the equivalent dermis before contraction;

“Si” represents the surface area of the equivalent dermis at the instant i in the contraction kinetics.

c) Results

As shown in the accompanying FIGURE, the degree of contraction of the control equivalent dermis was 32% four hours after having been detached from its support. It increased to 42% after eight hours and reached 54% after twenty-four hours.

Adenosine reduced this contraction percentage by 6.4% after four hours, 10.5% after eight hours and 12.7% after twenty-four hours compared with the control.

Thus, this test demonstrates that adenosine causes less contraction in an equivalent dermis, and thus has a relaxing effect which can be exploited in the preparation of compositions with a dermo-relaxant effect. As used herein, the relaxing effect is noted any time less contraction is observed, including less than 1%, 1%, 3%, 5%, etc.

### Example 2

### Cosmetic Composition

This composition was prepared in a manner that was conventional for the skilled person. The quantities given in this example are indicated as percentages by weight.

| | |
|---|---|
| Adenosine | 0.10% |
| Stearic acid | 3.00% |
| Mixture of glyceryl mono- | 2.50% |
| stearate and polyethylene | |
| glycol stearate (100 OE) | |
| Polyethylene glycol | 1.00% |
| stearate (20 OE) | |
| Cyclopentadimethylsiloxane | 10.00% |
| Fillers | 3.00% |
| Vegetable oils | 7.00% |
| Synthetic oils | 6.00% |
| Preservatives | 1.20% |
| Dimethylsiloxane, | 1.00% |
| oxyethylenated (16 OE) with | |
| methoxy extremities | |
| Silicone gum | 0.20% |
| Acrylic copolymer, in | 1.70% |
| reverse emulsion (Simulgel | |
| 600 from SEPPIC) | |
| Stearyl alcohol | 1.00% |
| Water | qsp |
| | 100% |

This cream was intended for application to the face and forehead to soften lines and relax the skin of the face.

The above written description of the invention provides a manner and process of making and using it such that any person skilled in this art is enabled to make and use the same, this enablement being provided in particular for the subject matter of the appended claims, which make up a part of the original description and including a cosmetic method for softening lines and/or relaxing the skin, and/or for relaxing facial features (“detendre les traits”) comprising topical application to the skin of a composition comprising at least one compound selected from adenosine and an analogue of adenosine, in a physiologically acceptable medium. Similarly, the invention composition can decrease wrinkles, reduce laugh lines, reduce frown lines, etc.

Preferred embodiments of the invention similarly fully described and enabled include use of at least one compound selected from adenosine and an adenosine analogue in a composition suitable for topical application to the skin, as an agent intended to soften lines and/or relax the skin, and the use of the invention compositions in an amount effective to provide a relaxing effect on contractile fibroblasts.

As used above, the phrases “selected from the group consisting of” and “selected from” include mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for softening lines and/or relaxing the skin and/or relaxing facial features, comprising topically applying to the skin a composition comprising at least one compound selected from the group consisting of adenosine and adenosine analogues, in a physiologically acceptable medium.

2. The method according to claim 1, wherein said composition comprises an adenosine analogue selected from the group consisting of: agonists of adenosine receptors, compounds increasing intra- or extra-cellular adenosine levels, and mixtures thereof.

3. The method according to claim 1, wherein said composition comprises at least one adenosine analogue selected from the group consisting of: 2′-deoxyadenosine; 2′,3′-isopropylidene adenosine; toyocamycin; 1-methyladenosine, N-6-methyladenosine; adenosine N-oxide; 6-methylmercaptopurine riboside; 6-chloropurine riboside; 5′-adenosine monophosphate; 5′-adenosine diphosphate and 5′-adenosine triphosphate; phenylisopropyl adenosine, 1-methylisoguanosine, N⁶-cyclohexyladenosine, N⁶-cyclopentyladenosine, 2-chloro-N⁶-cyclopentyladenosine, 2-chloroadenosine, N⁶-phenyladenosine, 2-phenylaminoadenosine, MECA, N⁶-phenethyladenosine, 2-p-(2-carboxyethyl)phenethyl-amino-5′-N-ethylcarboxamidoadenosine, N-ethylcarboxamidoadenosine, 5′-(N-cyclopropyl)-carboxamidoadenosine, DPMA and metrifudil; erythro-9-(2-hydroxy-3-nonyl) adenine and iodotubercidin.

4. The method according to claim 1, wherein the composition comprises 0.01% to 1% by weight of adenosine and/or adenosine analogue with respect to the total composition weight.

5. The method according to claim 2, wherein the composition comprises 0.01% to 1% by weight of adenosine and/or adenosine analogue with respect to the total composition weight.

6. The method according to claim 3, wherein the composition comprises 0.01% to 1% by weight of adenosine and/or adenosine analogue with respect to the total composition weight.

7. The method of claim 1, wherein the composition is applied to one or more zones of the face or forehead marked with expression lines and/or to persons having expression lines.

8. The method of claim 1, wherein said composition comprises adenosine.

9. The method of claim 4, wherein said composition comprises adenosine.

10. The method according to claim 1, comprising topically applying to the skin an amount of said composition effective to provide a relaxing effect on contractile fibroblasts.

11. The method according to claim 10, wherein said composition comprises an adenosine analogue selected from the group consisting of: agonists of adenosine receptors, compounds increasing intra- or extra-cellular adenosine levels, and mixtures thereof.

12. The method according to claim 10, wherein said composition comprises at least one adenosine analogue selected from the group consisting of: 2′-deoxyadenosine; 2′,3′-isopropylidene adenosine; toyocamycin; 1-methyladenosine, N-6-methyladenosine; adenosine N-oxide; 6-methylmercaptopurine riboside; 6-chloropurine riboside; 5′-adenosine monophosphate; 5′-adenosine diphosphate and 5′-adenosine triphosphate; phenylisopropyl adenosine, 1-methylisoguanosine, N⁶-cyclohexyladenosine, N⁶-cyclopentyladenosine, 2-chloro-N⁶-cyclopentyladenosine, 2-chloroadenosine, N⁶-phenyladenosine, 2-phenylaminoadenosine, MECA, N⁶-phenethyladenosine, 2-p-(2-carboxyethyl)phenethyl-amino-5′-N-ethylcarboxamidoadenosine, N-ethylcarboxamidoadenosine, 5′-(N-cyclopropyl)-carboxamidoadenosine, DPMA and metrifudil; erythro-9-(2-hydroxy-3-nonyl) adenine and iodotubercidin.

13. The method according to claim 10, wherein the composition comprises 0.01% to 1% by weight of adenosine and/or adenosine analogue with respect to the total composition weight.

14. The method according to claim 11, wherein the composition comprises 0.01% to 1% by weight of adenosine and/or adenosine analogue with respect to the total composition weight.

15. The method according to claim 12, wherein the composition comprises 0.01% to 1% by weight of adenosine and/or adenosine analogue with respect to the total composition weight.

16. The method of claim 10, wherein the composition is applied to one or more zones of the face or forehead marked with expression lines and/or to persons having expression lines.

17. The method of claim 10, wherein said composition comprises adenosine.

18. The method of claim 13, wherein said composition comprises adenosine.

19. The method of claim 1, wherein said composition comprises adenosine and at least one adenosine analogue.

20. The method of claim 10, wherein said composition comprises adenosine and at least one adenosine analogue.

21. The method of claim 1, comprising topically applying to the skin an effective amount of said composition to decrease wrinkles and/or reduce laugh lines and/or reduce frown lines.

22. The method of claim 8, comprising topically applying to the skin an effective amount of said composition to decrease wrinkles and/or reduce laugh lines and/or reduce frown lines.
